(19) United States
(12) Patent Application Publication
(10) Pub. No.: US 2021/0346718 A1
(43) Pub. Date: Nov. 11, 2021

(54) PHOTO-THERMAL TARGETED TREATMENT SYSTEM AND ASSOCIATED METHODS

(71) Applicant: Accure Acne, Inc., Boulder, CO (US)

(72) Inventor: Henrik Hofvander, Budapest (HU)

(73) Assignee: Accure Acne, Inc., Boulder, CO (US)

(21) Appl. No.: 17/315,112

(22) Filed: May 7, 2021

Related U.S. Application Data
(60) Provisional application No. 63/021,210, filed on May 7, 2020.

(51) Int. Cl. A61N 5/06

(52) U.S. Cl. CPC A61N 5/0625; A61B 2018/00791

(57) ABSTRACT

A method for operating a light source within a photo-thermal targeted treatment system for targeting a chromophore embedded within a medium is disclosed. The method includes: 1) applying a treatment protocol to a skin surface; 2) measuring a skin surface temperature while applying the treatment protocol; 3) calculating parameters regarding a heat transfer provided by the photo-thermal targeted treatment system based on the skin surface temperature so measured; and 4) adjusting the light source and the treatment protocol in accordance with the information regarding the heat transfer.

### FIELD OF THE INVENTION

The present invention relates to energy-based treatments and, more specifically, systems and methods for improving the safety and efficacy of an energy-based dermatological treatment.

### BACKGROUND OF THE INVENTION

Sebaceous glands and other chromophores embedded in a medium, such as the dermis, can be thermally damaged by heating the chromophore with a targeted light source, such as a laser. However, the application of enough thermal energy to damage the chromophore can also be damaging to the surrounding dermis and the overlying epidermis, thus leading to epidermis and dermis damage as well as pain to the patient.

Previous approaches to prevent epidermis and dermis damage as well as patient pain during a photo-thermal treatment procedure include:

1. Cooling the epidermis, then applying the photo-thermal treatment; and

2. Cool the epidermis, also condition (i.e., preheat) the epidermis and dermis in a preheating protocol, then apply photo-thermal treatment in a distinct treatment protocol. In certain instances, the preheating protocol and the treatment protocol are performed by the same laser, although the two protocols would involve different laser settings and application protocols, thus leading to further complexity in the treatment protocol and equipment.

For either of these approaches, as well as in many energy-based dermatological procedures, measuring the temperature of the skin surface during the treatment provides valuable information that can be used to adjust the treatment protocol and/or equipment settings in real time. Such temperature-based treatment protocol adjustments can be made by a practitioner, as an example, every few seconds over an hour-long procedure or can be made automatically by the system itself in a closed-loop control fashion. However, any analytics of the underlying tissue based on skin surface measurements, or the use of skin surface temperature measurements to either suggest changes in settings or to make such adjustments automatically require a number of assumptions, such as the heat transfer coefficient and fluence (i.e., optical energy delivered per unit area), and these factors differ from system to system, even patient to patient. Particularly in photo-thermal targeted treatment systems involving both cooling and heating of the treatment area, inaccuracies in these assumed parameters can greatly influence the actual performance of the system during application of the treatment protocol.

### SUMMARY OF THE INVENTION

In accordance with the embodiments described herein, there is described a method for operating a light source within a photo-thermal targeted treatment system for targeting a chromophore embedded within a medium. The method includes: 1) applying a treatment protocol to a skin surface; 2) measuring a skin surface temperature while applying the treatment protocol; 3) calculating parameters regarding a heat transfer provided by the photo-thermal targeted treatment system based on the skin surface temperature so measured; and 4) adjusting the light source and the treatment protocol in accordance with the information regarding the heat transfer.

In accordance with another embodiment, a photo-thermal targeted treatment system is described. The photo-thermal targeted treatment system includes a light source for providing a light output toward a treatment area, a cooling unit for providing a cooling mechanism at the treatment area, a temperature monitoring unit for measuring a skin surface temperature at the treatment area to provide a skin surface temperature measurement, and a controller for controlling the operating parameters of the light source, the cooling unit, and the temperature monitoring unit. The controller is configured for receiving the skin surface temperature measurement, calculating at least one heat transfer parameter of the photo-thermal targeted treatment system based on the skin surface temperature measurement, and adjusting at least one of the light source and the cooling unit, in accordance with the at least one heat transfer parameter so calculated.

### BRIEF DESCRIPTION OF THE DRAWINGS

FIG. 1 shows an exemplary photo-thermal targeted treatment system for targeting a target, wherein the target includes specific chromophores embedded in a medium, and heating the target to a sufficiently high temperature so as to damage the target without damaging the surrounding medium. The system can be used, for example, for photo-thermal ablation of sebaceous glands in a targeted fashion, where sebum is the chromophore embedded within the sebaceous gland, while sparing the epidermis and dermis surrounding the target sebaceous glands.

FIG. 2 shows the measured temperature at the skin surface as a function of time as the treatment photo pulses are applied thereto, in accordance with an embodiment.

FIG. 3 shows the envelope fits for measured low and high peak epidermal temperature (i.e., skin surface temperature) readings over 45 trigger pulls, in accordance with an embodiment.

FIG. 4 shows the measured temperature at the skin surface as a function of time as the treatment photo pulses are applied thereto, along with a curve fit of the skin surface temperature as cooling is applied thereto prior to the application of the treatment photo pulses, in accordance with an embodiment.

FIG. 5 shows the measured temperature at the skin surface as a function of time as the treatment photo pulses are applied thereto, along with a curve fit of the skin surface temperature during the cooldown period between the application of treatment photo pulses, in accordance with an embodiment.

### DETAILED DESCRIPTION OF EMBODIMENTS OF THE INVENTION

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity. Like numbers refer to like elements throughout.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items, and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

When performing a dermatological treatment, it is preferable that the practitioner keeps his/her eyes continuously on the current treatment location. The reasons include:

1. Safety
A. The practitioner can ensure that the system is performing correctly by viewing, for example, an aiming beam that is in the visual range
B. The practitioner can ensure that there are no adverse events, e.g., blisters on the skin surface
C. The practitioner can ensure that no two treatment areas are being overlapped, i.e., the same area is being treated twice. Anytime the practitioner looks away from the treatment area, there is a risk the s/he loses track of where the last treatment took place and where to go to next.

2. Efficacy: The practitioner can ensure that no treatment areas are being skipped. For instance, keeping the practitioner's attention at the treatment location makes it less likely that the practitioner will lose track of where the next treatment location is if s/he is following a grid pattern.

3. Ergonomics: Having to constantly swivel his/her head to look at a screen is not a good ergonomic setup.

4. Economics: Currently, an assistant is required in the treatment room to call out temperatures, even if the temperature is displayed on the treatment system user interface. Having an assistant attend the procedure, even if the assistant is not required in the use of the treatment system, adds to the cost of operating the treatment system.

In laser treatment of acne, the operating thermal range is generally bound on the upper end at the epidermis and dermis damage threshold temperature of approximately 55° C., and at the lower end by the temperature required to bring the sebaceous gland to its damage threshold temperature of approximately 55° C. Based on clinical data, the operating temperature range for acne treatment expressed in terminal skin surface temperature is approx. 45° C. to 55° C., as an example. At skin surface temperatures below 45° C., it has been determined that there is no damage to the sebaceous gland. When the skin surface temperature is between 45° C. and 55° C., there are varying degrees of sebaceous gland damage, with no epidermal damage. Above 55° C., there is epidermal damage in addition to damage to the sebaceous gland. By adjusting the treatment system, such as the light source operating parameter settings, in real time during treatment, the practitioner can avoid damaging the epidermis and dermis surrounding the treatment area, while effectively administering the treatment protocol.

Exemplary System

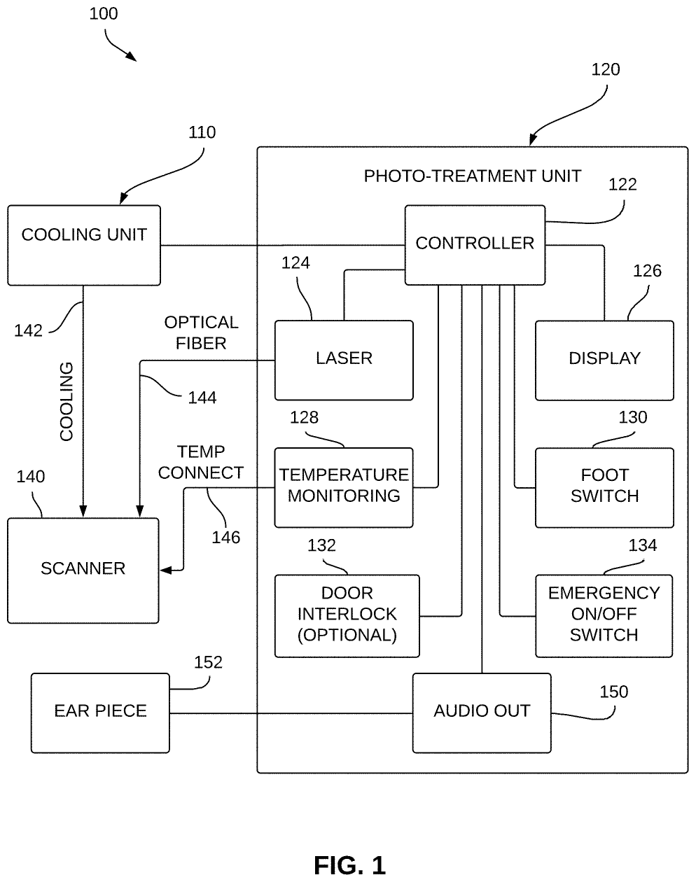

FIG. 1 shows an exemplary photo-thermal targeted treatment system for targeting a target, wherein the target includes specific chromophores embedded in a medium, and heating the target to a sufficiently high temperature so as to damage the target without damaging the surrounding medium. The system can be used, for example, for photo-thermal ablation of sebaceous glands in a targeted fashion, where sebum is the chromophore embedded within the sebaceous gland, while sparing the epidermis and dermis surrounding the target sebaceous glands.

Still referring to FIG. 1, a photo-thermal targeted treatment system 100 includes a cooling unit 110 and a photo-treatment unit 120. Cooling unit 110 provides a cooling mechanism for a cooling effect, such as by contact or by direct air cooling, to treatment area, namely the outer skin layer area overlying the target sebaceous gland. Cooling unit 110 is connected with a controller 122 within photo-treatment unit 120. It is noted that, while controller 122 is shown to be contained within photo-treatment unit 120 in FIG. 1, it is possible for the controller to be located outside of both cooling unit 110 and photo treatment unit 122, or even within cooling unit 110.

Controller 122 further controls other components within photo-treatment unit 120, such as a laser 124, a display 126, a temperature monitoring unit, a foot switch 130, a door interlock 132, and an emergency on/off switch. Laser 124 provides the laser power for the photo-treatment protocol, and controller 122 regulates the specific settings for the laser, such as the output power and pulse time settings. Laser 124 can be a single laser or a combination of two or more lasers. If there more than one laser is used, the laser outputs are combined optically to function as one more powerful laser. Display 126 can include information such as the operating conditions of cooling unit 110, laser 124, and other system status. Temperature monitoring unit 128 is used to monitor the temperature of the skin surface in the treatment area, for example, and the measured skin surface temperature at the treatment area is used by controller 122 to adjust the photo-treatment protocol. Controller 122 also interfaces with footswitch 130 for remotely turning on or off laser 124 and/or cooling unit 110. Additionally, door interlock 132 can be used as an additional safety measure such that, when the door to the treatment room is ajar, door interlock 132 detects the condition and instructs controller 122 to not allow photo-treatment unit 120, or at least laser 124, to operate. Furthermore, emergency on/off switch 134 can be provided to quickly shut down photo-thermal targeted treatment system 100 in case of an emergency. In another modification, additional photodiodes or other sensors can be added to monitor the power level of the energy emitted from laser 124.

Continuing to refer to FIG. 1, photo-thermal targeted treatment system 100 further includes a scanner 140, which is the portion of the device handheld by the user in applying the treatment protocol to the subject. Scanner can be formed, for example, in a gun-like or stick-like shape for ease of handling by the user. Scanner 140 is connected with cooling unit 110 via a cooling connection 142, such that the cooling protocol can be applied using scanner 140. Additionally, the output from laser 124 is connected with scanner 140 via an optical fiber delivery 144, such that the photo-treatment protocol can be applied using scanner 140. Scanner 140 is connected via a temperature connection 146 to temperature monitoring unit 128, so as to feedback the skin temperature at the treatment area, for example, to controller 122.

Additionally, photo-treatment unit 120 may further include an audio out circuitry 150 for providing an audio output, such as a skin surface temperature reading as recorded at temperature monitoring unit 128. Audio out circuitry 150 provides a signal to, for example, an ear piece 152 through a wired or wireless connection such that the practitioner using the system can listen to the audio output. Ear piece 152 can be replaced, for instance, by a speaker system or other audio communication means. Audio out circuitry can also convey other information such as the status of the photo-treatment unit, any emergency warnings, or other messages to be conveyed to the user of photo-thermal targeted treatment system 100.

Many laser treatment systems rely on a thermal balance provided by thermal energy input via a laser and thermal energy removal via a cooling mechanism. In order to optimize both the safety and efficacy of such laser treatment systems, it would be desirable to have close control over both sides of this thermal balance equation. While the thermal energy input via the laser is relatively simple to measure and adjust via a closed-loop control system, cooling systems more challenging to control. The amount of thermal energy removal provided by a cooling mechanism, particularly air cooling systems, is difficult to quantify. Ideally, a measurement of the heat transfer (i.e., how much heat in W or J is being removed) provided by the cooling mechanism would be desirable.

System Fluence Metric T_Rise/W

Skin surface temperature measurements of the treatment area during application of the treatment protocol can assist in the prevention of patient injury while improving efficacy of the treatment. However, in evaluating the actual performance of the photo-thermal targeted treatment system in use, it is difficult to separate the thermal effects of cooling and fluence. Especially given the interwoven nature of the cooling and heating mechanisms in a photo-thermal targeted treatment system such as shown in FIG. 1, an accurate measure of the heat extraction of the cooling system is required to both analyze the cooling system as well as analyze the system fluence.

Rather than directly using the raw skin surface measurements, we recognize herein that the rise temperature T_rise, the epidermal temperature rise upon delivery of a treatment pulse, per watt (W) of energy delivered by the treatment pulse (i.e., T_rise/W) provides a suitable metric for fluence or a particular photo-thermal targeted treatment system, irrespective of the heat transfer coefficient.

Estimating System Heat Transfer Coefficient

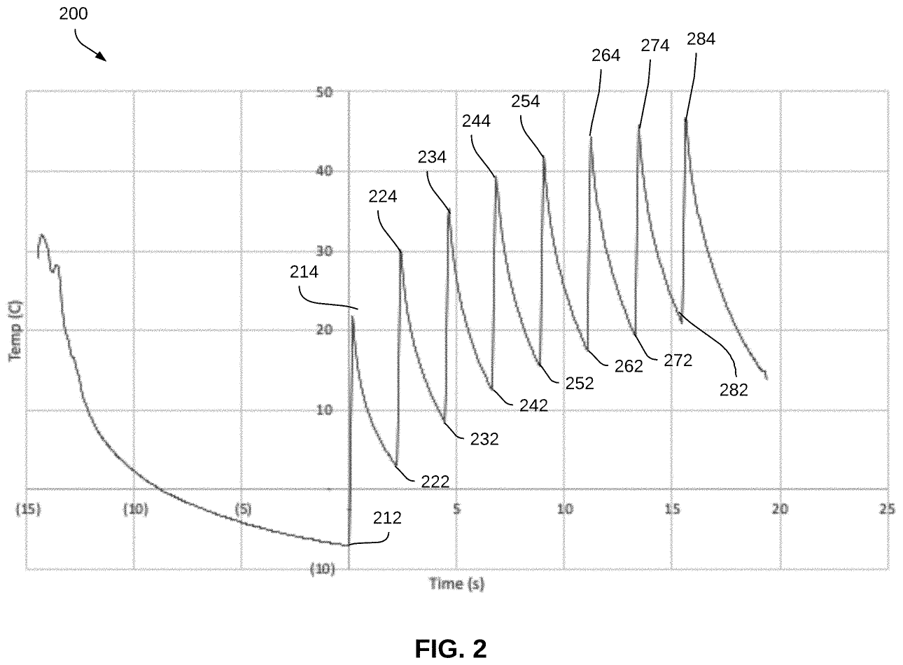

Referring now to FIG. 2, a graph 200 shows the measured skin surface temperature as eight light pulses are applied to the treatment area, in accordance with an embodiment. In the example shown in FIG. 2, the treatment area had been precooled by direct air cooling for 14 seconds, then light pulses from a 1726 nm wavelength laser at 22 watts power and 150 milliseconds in duration were applied with a period of 2.1 seconds, while the cooling remains on. In this particular example, the direct-air cooling used for the cooling and during the treatment, delivers a high speed column of air, cooled to −18° C., resulting in a heat transfer coefficient between the skin and the air of approximately 500 W/m\hat{ }2 K. The beam size is 4.0 mm square and the average power per spot is 22 W*0.15 s/2.1 s=1.57 W, in an embodiment. The exact beam size can be adjusted, using for example collimation optics, depending on the size of the treatment area, power profile of the laser, the location of the treatment area of the body, and other factors. The skin surface temperature measurements, in the example shown, are performed using an infrared (IR) camera.

The resulting changes in skin surface temperature are shown in graph 200. As shown in FIG. 2, the skin surface temperature at the application of the first pulse at time zero is approximately −7° C. (indicated by a nadir 212), and rises to approximately 22° C. upon application of a first treatment pulse (indicated by a peak 214). An additional seven pulses are then applied, as shown in graph 200 to result in nadir-peak combinations 222-224, 232 234, 242-244, 252-254, 262-264, 272-274, and 282-284.

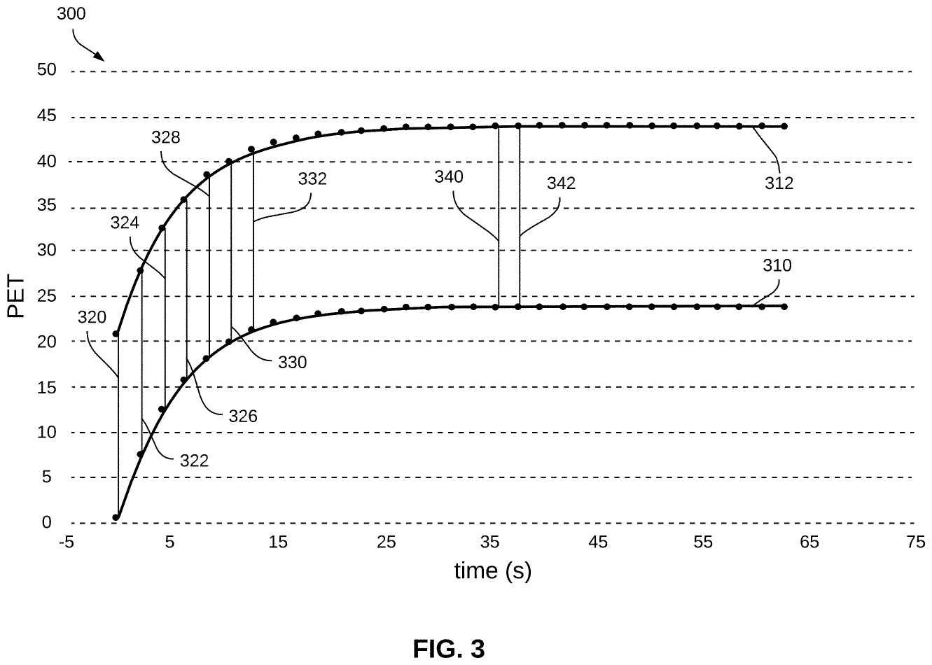

The nadir-peak combinations can be alternatively shown as in FIG. 3, which shows a graph 300 indicating the skin surface temperature nadir values (shown as line 310, connecting the nadir values in a curve fit) and skin surface temperature peak values (shown as line 312, connecting the peak values in a curve fit) over time. The curve can be extrapolated using the application of additional virtual pulses beyond the eight pulses indicated in FIG. 2 until the system reaches thermal equilibrium. The extrapolation can be shown to be valid by how good the curve fit is for the 8 peaks and nadirs and it can also be valid by finite element heat transfer modeling. In both cases, an exponential fit is used in the present example. The nadir and peak values for each pulse for the first seven pulses are connected by lines 320, 322, 324, 326, 328, 330, and 332, respectively. Lines 340 and 342 connect the nadir and peak values for virtual pulses 18 and 19. The difference between the nadir and peak values for each pulse is referred to as the T_rise value:

T_rise=T_peak−T_nadir [Eq. 1]

For the first seven pulses, the T_rise values are summarized in Table 1 below, where the average of the T_rise(° C.) values for all pulses is considered T_rise (%)=100%:

TABLE 1

| Time (s) | T_nadir (° C.) | T_peak (° C.) | T_rise (° C.) | T_rise (%) |
|---|---|---|---|---|
| 0 | 0.32 | 20.78 | 20.46 | 100.5 |
| 2.1 | 7.26 | 27.71 | 20.46 | 100.5 |
| 4.2 | 12.14 | 32.56 | 20.42 | 100.3 |
| 6.3 | 15.59 | 35.95 | 20.37 | 100.1 |
| 8.4 | 18.01 | 38.32 | 20.31 | 99.8 |
| 10.5 | 19.72 | 39.98 | 20.26 | 99.5 |
| 12.6 | 20.93 | 41.14 | 20.22 | 99.3 |

It can be seen in Table 1 that T_rise is, to first order, independent of T_nadir and T_peak values and the derivation below will show that it is also virtually independent of any Heat Extraction, HE, effects. The HE can be characterized as a factor of the system Heat Transfer Coefficient (HTC), average skin surface temperature T_ave, and the air temperature T_air:

HE˜HTC*(T_ave−T_air) [Eq. 2]

Specifically related to the treatment system evaluated in FIGS. 2 and 3 as well as Table 1, T_air is −21° C. For the first pulse at t=0, the average temperature during the pulse is:

T_ave1=(T_peak+T_nadir)/2=(20.78° C.+0.32° C.)/2=10.55° C. [Eq. 3]

Cooling (heat extraction) is proportional to the heat transfer coefficient:

HE1=HTC*(10.55° C.−(−21° C.))=HTC*31.55° C. [Eq. 4]

Similarly for pulse 7 at t=12.6 seconds:

$$\begin{matrix}\begin{matrix}T\_ave7 = ( T\_peak + T\_nadir ) / 2 \\ = ( 41.14 ° C + 20.93 ° C ) / 2 = 31.03 ° C\end{matrix} & [ Eq . 5 ] \\ HE 7 = HTC * ( 31.02 ° C - ( - 21 ° C ) ) = HTC * 52.02 ° C & [ Eq . 6 ]\end{matrix}$$

Despite there being a difference of 52.02° C./31.55° C.=65% in cooling between pulse 7 and pulse 1, T_rise for pulse 1 and pulse 7 differs by less than 1%. Thus, we assert that T_rise is for practical purposes independent of HTC.

In the above calculations, it is assumed that the cooling (i.e., heat extraction) is dominated by the cooling air provided to the skin surface, not diffusion into other parts of the tissue. We also assume the pulse itself is short, e.g., 150 milliseconds, limiting the effect of any diffusion from the skin tissue at the treatment site. Additionally, we assume the average temperature during the pulse is a close approximation of the actual temperature behavior. While we are aware the actual temperature behavior is an exponential decay, using the average value is consistent across the various pulses for the purposes of the present calculations.

Thought of in another way, in FIG. 3, the treatment area would reach a thermal equilibrium at around t=25 seconds or pulse 13. That is, at this point, the cooling power and heating power (i.e., laser power provided by the laser) are in balance:

P_laser=P_cooling [Eq. 7]

Similarly, the laser energy of the treatment pulse is delivered during the 150 ms pulse duration, and the cooling energy is delivered by the cooling mechanism in a uniform manner over the full period of 2.1 seconds. That is, over a full period of 2.1 seconds, including the applied pulse duration plus the period between treatment pulse applications, the laser energy would be equal to the cooling energy provided by the cooling mechanism:

E_laser=E_cooling [Eq. 8]

Thus, the cooling energy would only affect the treatment pulse only during the treatment pulse period of 150 ms, thus any change in cooling would only affect the pulse by a ratio of 150 ms/2.1 s=0.075 (i.e., 7.5%). Therefore, to a first order, moderate variations in HTC would only minimally affect the energy delivered in a treatment pulse and, thereby, variation in HTC would not affect T_rise to an appreciable degree.

One aspect of the treatment protocol which should be taken into account is the laser spot size, as the temperature sampling area to measure T_rise is sampled over a known area size. For instance, in using an IR camera to measure skin surface temperature, the temperature of a skin surface area corresponding to an area of 3.2 mm by 3.2 mm is measured, in accordance with an embodiment. Thus, T_rise/W is an appropriate metric for laser fluence as the area over which the skin surface temperature is measured is known, and any variance in the actual laser spot size and shape from system to system is irrelevant.

Relative Heat Transfer Coefficient h′

In order to more accurately compare and predict the performance of different systems, it would be desirable to have an understanding of the Heat Extraction, HE, characteristic of each system. While the actual HE value for each system is difficult to ascertain, a relative heat transfer coefficient, h′, can be calculated as a measure of the Heat Transfer Coefficient (HTC) for a single system. While the relative HTC (h′) does not allow comparison of HTC values between different systems, because it does not take into account variations in spot sizes and shapes delivered at the treatment area, h′ does allow the user to evaluate the performance of a given photo-thermal targeted treatment system with a known spot size and shape, especially over time. In other words, even without precise knowledge of the laser spot size and shape, since the way the laser deposits its energy is essentially constant with each laser pulse, h′ is a valid measurement for evaluating a given treatment system.

The value of the relative HTC h′ for a given system can be derived using the following method, for example. As shown in Eqs. 7 and 8, at thermal equilibrium, it is recognized that the heating power provided by the laser and the cooling power provided by the cooling mechanism are in balance such that the laser input power is equal to the cooling power. Similarly, over a full period of 2.1 seconds, the energy provided by the laser is equal to the energy provided by the cooling mechanism. Thus, we can conclude that, at thermal equilibrium,

E_cooling˜HTC*Temperature differential=HTC*∫[Tₛₖᵢₙ−Tₐᵢᵣ] [Eq. 9]

where the integral is over the time period of one pulse. As above, the integral over the skin temperature Tₛₖᵢₙ is approximated by the average temperature of the peak and nadir temperatures for a given pulse. For example, for the pulse around 35 s in FIG. 3 (indicated by line 340), T_peak is 43.78° C. and T_nadir is 23.74° C., such that the average temperature T_ave is

(T_peak+T_nadir)/2=(43.78° C.+23.74° C.)/2=33.76° C. [Eq. 10]

Consequently, cooling energy can be calculated:

E_cooling=HTC*Area_cooling*full period*(T_skin−T_air) [Eq. 11]

where T_air is the measured temperature of the cooling air in the case of an air-cooled system.

In the illustrated example, the laser energy is E_laser=23 W*150 ms over an unknown yet constant spot area, and T_air is −21° C. We assume the cooling area is much larger than the laser spot, and the cooling provided by the cooling mechanism is uniform. Thus, at thermal equilibrium:

E_laser=E_cooling [Eq. 12]

→23 W*0.15 s=HTC*Area_cooling*2.1 s*(33.76° C.−(−21° C.)) [Eq. 13]

Solving for HTC*Area_cooling, we find:

HTC*Area_cooling=0.030=h′ [Eq. 14]

The relative HTC, h′, is a valuable metric in evaluating the performance of the photo-thermal targeted treatment system. For instance, h′ provides a direct measurement to infer the HTC for a particular system, thus allowing the application of closed-loop control for in situ adjustment of the cooling mechanism. As an example, a closed-loop control system can be implemented to control the operations of the cooling mechanism in order to compensate for any system performance variations due to environmental factors (e.g., temperature, humidity, altitude) and system factors (e.g., restriction of intake air due to frosting). Similarly, any other changes in the cooling mechanism performance can be quantified using h′.

Heat Extraction Rate Control

A First Alternate Approach to Estimating System Heat Transfer Coefficient

The heat transfer can be somewhat estimated by measuring the temperature of the cooling air (e.g., using a thermistor) and the air velocity at the air outlet of the cooling mechanism (e.g., using a heated pitot tube). However, variations in environmental factors (e.g., altitude, ambient temperature, ambient humidity, patient temperature at the treatment area) often lead to inaccuracies.

Newton's law of cooling formula is

T=T_equilibrium+(T_initial−T_equilibrium)*exp(−k*t) [Eq. 15]

where:
T [in Kelvins] is the temperature of the object at the time t,
T_equilibrium [K] is temperature that the object will reach if the pulsing protocol where to last until a thermal equilibrium is reached
T_initial [K] is the initial temperature of the object
k [l/s] is the cooling coefficient, and
t [s] is the time duration of the cooling applied to the object.

How quickly the object cools down depends on two factors. The first factor is the difference in the temperatures between the object (e.g., skin tissue) and the cooling medium (e.g., cold air applied to the skin). The larger the difference, the quicker the cooling. The second factor is the cooling coefficient k, which depends on the mechanism of the cooling and the amount of heat that is exchanged. The cooling coefficient can be expressed as:

k=h*A/C, [Eq. 16]

where:
k [l/s] is the cooling coefficient,
h [W/(m²*K)] is the heat transfer coefficient,
A [m²] is the area of the heat exchange,
C [J/K] is the heat capacity.
   Heat capacity calculations can be found, at websites such as: https://www.omnicalculator.com/physics/specific-heat

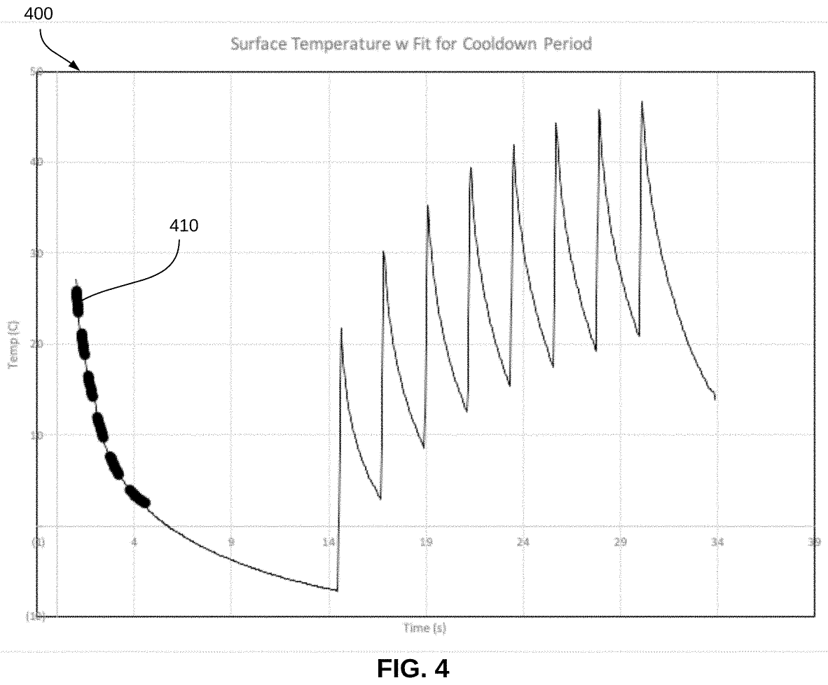

We recognize herein that the fall time of the skin surface temperature from the onset of the pre-cooling period (e.g., as shown between time −15 to 0 second in FIG. 2 or from time 0 to 15 seconds in graph 400 of FIG. 4) can be expressed as an exponential or a series of exponentials following the formula given above:

T=T_equilibrium+(T_initial−T_equilibrium)*exp(−k*t) [Eq. 17]

From the curve fit of Eq. 17, shown as a thick dashed line 410 in FIG. 4, we can directly solve for k, the cooling coefficient.

A Second Alternate Approach to Estimating System Heat Transfer Coefficient

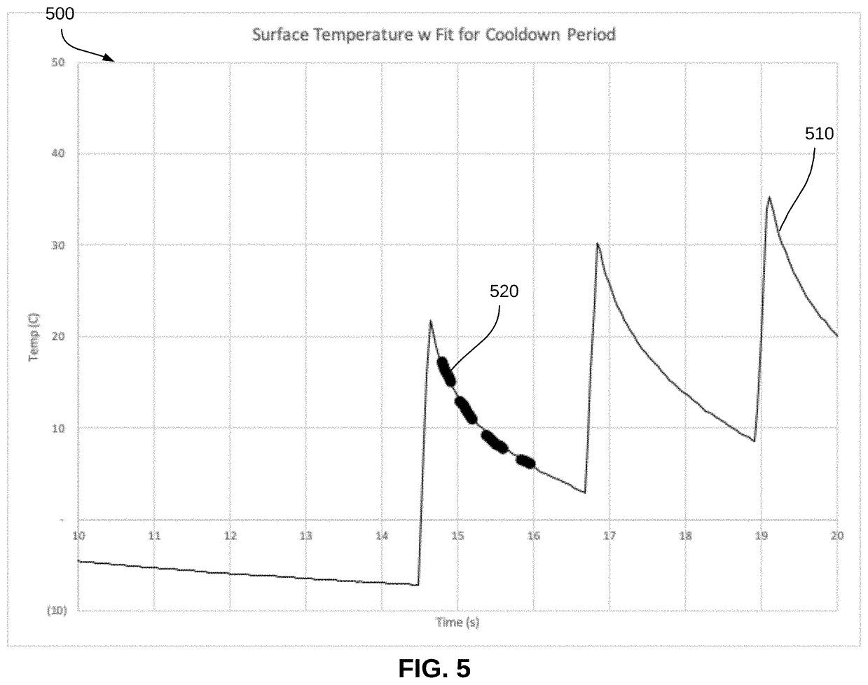

Using the same equations as above, but using the time interval between the pulses as opposed to the pre-cooling period, we can again estimate k, the cooling coefficient. A graph 500 in FIG. 5 shows the first few pulses of a pulsing protocol, as represented by a plot 510. A curve fit using Eq. 17 can be used during a time interval between pulses (e.g., represented by a thick dashed line 520 in FIG. 5 for time 14.5 seconds to 16 seconds) then the value for k can be derived.

Knowledge regarding h′ and k k, since both are direct metric of the Heat Extraction for a given photo-thermal targeted treatment system, can be directly used as an input for a closed-loop control of the cooling system. For instance, any changes in h′ can be used as an indicator of system changes, including cooling, over time. Thus, the information so derived above based on specific measurements and parameters of the particular photo-thermal treatment system and its use conditions can help protect the safety of the patient in ways heretofore unavailable to the system user. For example, the information so derived above can be used to adjust the cooling setting of cooling unit 110, power, pulse width, duty cycle or other operating parameters of laser 124, and other settings of system 100 as shown in FIG. 1.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

For example, embodiments such as the below are contemplated:

1. A method for operating a light source within a photo-thermal targeted treatment system for targeting a chromophore embedded within a medium. The method includes: 1) applying a treatment protocol to a skin surface; 2) measuring a skin surface temperature while applying the treatment protocol; 3) inferring information regarding a heat transfer provided by the photo-thermal targeted treatment system; and 4) adjusting the light source and the treatment protocol in accordance with the information regarding the heat transfer.

2. The method of Item 1, wherein the information regarding the heat transfer is a system fluence metric.

3. The method of Item 1, wherein the information regarding the heat transfer is a relative heat transfer coefficient.

4. The method of Item 1, wherein the information regarding the heat transfer is a heat extraction rate.

In the specification, there have been disclosed embodiments of the invention and, although specific terms are employed, they are used in a generic and descriptive sense only and not for purposes of limitation. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the claimed invention

What is claimed is:

1. A method for operating a light source within a photo-thermal targeted treatment system, the method comprising:
applying a first round of a treatment protocol to a skin surface;
measuring a temperature at the skin surface while applying the treatment protocol thereto;
calculating at least one heat transfer parameter of the photo-thermal targeted treatment system based on the temperature so measured;
adjusting at least one of the light source and the treatment protocol, in accordance with the at least one heat transfer parameter; and
applying a second round of the treatment protocol to the skin surface.

2. The method of claim 1, wherein the at least one heat transfer parameter includes at least one of a system heat transfer coefficient, a relative heat transfer coefficient, a heat extraction rate, and a cooling coefficient.

3. The method of claim 1,
wherein the photo-thermal targeted treatment system further includes a cooling unit, a temperature monitoring unit, and a controller for controlling the light source, cooling unit, and temperature monitoring unit,
wherein measuring the temperature at the skin surface includes measuring the temperature using the temperature monitoring unit, and
wherein adjusting at least one of the light source and the treatment protocol includes adjusting an operating parameter of at least one of the light source and the cooling unit prior to applying the second round of the treatment protocol to the skin surface.

4. A photo-thermal targeted treatment system, comprising:
a light source for providing a light output toward a treatment area;
a cooling unit for providing a cooling mechanism at the treatment area;
a temperature monitoring unit for measuring a skin surface temperature at the treatment area to provide a skin surface temperature measurement; and
a controller for controlling the operating parameters of the light source, the cooling unit, and the temperature monitoring unit,
wherein the controller is configured for receiving the skin surface temperature measurement, calculating at least one heat transfer parameter of the photo-thermal targeted treatment system based on the skin surface temperature measurement, and adjusting at least one of the light source and the cooling unit, in accordance with the at least one heat transfer parameter so calculated.
